(19) United States
(12) Patent Application Publication
(10) Pub. No.: US 2004/0110954 A1
(43) Pub. Date: Jun. 10, 2004

(54) Methods of synthesizing phenol-contining compounds

(76) Inventors: Michael R. Palovich, Collegeville, PA (US); Katherine L. Widdowson, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/473,105

(22) PCT Filed: Mar. 27, 2002
(86) PCT No.: PCT/US02/10038

(51) Int. Cl. C07C 311/27; C07D 207/46

(52) U.S. Cl. 546/232; 548/542; 564/79; 564/93; 562/430

(57) ABSTRACT

Methods of synthesizing phenol-containing compounds are disclosed.

### FIELD OF THE INVENTION

This invention relates to the method of placing a sulfone or sulfonamide group ortho to a phenol in a drug substance in order to increase the metabolic stability and the half-life of the compound, while maintaining the acidity of the phenol.

### BACKGROUND OF THE INVENTION

Phenols are often found to be important pharmacophores for a number of target receptors, such as interleukin-8, opioid, dopamine, seritonin, COX1, COX2, andrenergic, and estrogen receptors. They are also found in a number of enzyme inhibitors such as betalactamases and topoisomerases. However the utility of drugs containing phenols is often limited by the short half-lives of these compounds due to conjugative metabolism via glucuronidation and/or sulfation of the phenol (see Mulder, G. J. and Meerman, J. H. Conjugative Reactions in drug Transformation edited by A. Aito (Amsterdam: Elseveir-North Holland), pp 389-397, 1978 also see Silverman, R. B. The Organic Chemistry of Drug Design and Drug Action, p 327-333, 1992). For example morphine, which contains a phenol, has a short half-life and high first pass clearance which limits it to intravenous administration. The major metabolite of phenol containing drugs such as morphine, acetaminophen and albuterol is glucuronidation or sulfation of the phenol (PDR).

There have been some studies comparing the rates of glucuronidation and sulfation in vivo and in vitro of various substituted phenols (E. Holmes Xenobiotica, 1995, 25(12), 1269-1281 and A. Timellini Xenobiotica, 1991, 21(2), 171-177). However, these studies do not specifically mention either sulfonamides or sulfones, nor do they explain why such functional groups would be so effective at blocking glucuronidation. Data presented in the paper written by A. Temellini on the structural activity relationship. of human liver sulfotranferase and glucuronidase suggests that extremely bulky substituents ortho to the phenol such as t-butyl appear to inhibit glucuronidation but an electron-withdrawing group such as nitro seems to increase glucuronidation rates. This would suggest that bulky alkyl substituents such as t-butyl would be effective in decreasing glucuronidation rates, but sulfonamides or sulfones would not be as effective since these groups are more electron withdrawing.

### SUMMARY OF THE INVENTION

This invention relates to the method of placing a sulfone or sulfonamide group ortho to a phenol in a drug substance in order to increase the metabolic stability and the half-life of the compound, while maintaining the acidity of the phenol.

Compounds of Formula (1) useful in the present invention are represented by the structure:

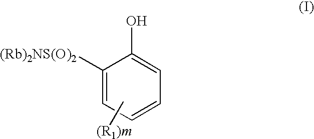

wherein:

R_{b} is independently selected from the group consisting of hydrogen, NR₆R₇, OH, ORₐ, C₁₋₅alkyl, aryl, aryl C₁₋₄alkyl, aryl C₂₋₄ alkenyl, cycloalkyl, cycloalkyl C₁₋₅ alkyl, heteroaryl, heteroarylC₁₋₄ alkyl, heteroaryl C₂₋₄ alkenyl, heterocyclic, heterocyclic C₁₋₄ alkyl, and a heterocyclic C₂₋₄ alkenyl moiety, all of which moieties may be optionally substituted one to three times independently by a substituent selected from the group consisting of halogen, nitro, halosubstituted C₁₋₄ alkyl, C₁₋₄ alkyl, amino, mono or di-C₁₋₄ alkyl substituted amine, ORₐC(O)Rₐ, NRₐC(O)ORₐ, OC(O)NR₆R₇, hydroxy, NR₉C(O)Rₐ, S(O)ₘ′Rₐ, C(O)NR₆R₇, C(O)OH, C(O)ORₐ, S(O)₂NR₆R₇, and NHS(O)₂Rₐ; or the two R_{b} substituents can join to form a 3-10 membered ring, optionally substituted and containing, in addition to carbon, independently, 1 to 3 substituents selected from the group consisting of NRₐ, O, S, SO, and SO₂, which substituents can be optionally unsaturated;

R₁ is independently selected from the group consisting of hydrogen, halogen, nitro, cyano, C₁₋₁₀ alkyl, halosubstituted C₁₋₁₀ alkyl, C₂₋₁₀ alkenyl, C₁₋₁₀ alkoxy, halosubstituted C₁₋₁₀ alkoxy, azide, S(O)ₜR₄, (CR₈R₈)_{q} S(O)ₜR₄, hydroxy, hydroxy substituted C₁₋₄ alkyl, aryl, aryl C₁₋₄ alkyl, aryl C₂₋₁₀ alkenyl, aryloxy, aryl C₁₋₄ alkyloxy, heteroaryl, heteroaryl alkyl, heteroaryl C₂₋₁₀ alkenyl, heteroaryl C₁₋₄ alkyloxy, heterocyclic, heterocyclic C₁₋₄ alkyl, heterocyclic C₁₋₄ allyloxy, heterocyclic C₂₋₁₀ alkenyl, NR₄C(O)NR₄R₅, NR₄C(S)NR₄R₅, (CR₈R₈)_{q} NR₄R₅, (CR₈R₈)_{q}C(O)NR₄R₅, C₂₋₁₀ alkenyl C(O)NR₄R₅, (CR₈R₈)_{q} C(O)NR₄R₁₀, S(O)₃R₈, (CR₈R₈)_{q} C(O)R₁₁, C₂₋₁₀ alkenyl C(O)R₁₁, C₂₋₁₀ alkenyl C(O)OR₁₁, (CR₈R₈)_{q} C(O)OR₁₁, (CR₈R₈)_{q} OC(O)R₁₁, (CR₈R₈)_{q}NR₄C(O)R₁₁, (CR₈R₈)_{q} C(NR₄)NR₄R₅, (CR₈R₈)_{q} NR₄C(NR₅)R₁₁, (CR₈R₈)_{q} NHS(O)₂R₁₃, (CR₈R₈)_{q} S(O)₂NR₄R₅, and

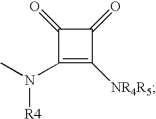

or two R₁ moieties together may form O—(CH₂)ₛO or a 5 to 6 membered saturated or unsaturated ring, wherein the alkyl, aryl, arylalkyl, heteroaryl, heterocyclic moieties may be optionally substituted;

R₄ and R₅ are independently selected from the group consisting of hydrogen, optionally substituted C₁₋₄ alkyl, optionally substituted aryl, optionally substituted aryl C₁₋₄ alkyl, optionally substituted heteroaryl, optionally substituted heteroaryl C₁₋₄ alkyl, heterocyclic, and heterocyclic C₁₋₄ alkyl; or R₄ and R₅ together with the nitrogen to which they are attached form a 5 to 7 member ring which may optionally comprise an additional heteroatom selected from oxygen, nitrogen and sulfur;

R₆ and R₇ are independently selected from the group consisting of hydrogen, C₁₋₄ alkyl, heteroaryl, aryl, aklylaryl, and alkyl C₁₋₄ heteroalkyl; or R₆ and R₇ together with the nitrogen to which they are attached form a 5 to 7 member ring which ring may optionally contain an additional heteroatom is selected from oxygen, nitrogen or sulfur, and which ring may be optionally substituted;

Rₐ is selected from the group consisting of alkyl, aryl, aryl C₁₋₄ alkyl, heteroaryl, heteroaryl C₁₋₄alkyl, heterocyclic, COORₐ, and a heterocyclic C₁₋₄ alkyl moiety, all of which moieties may be optionally substituted;

R₈ is hydrogen or C₁₋₄ alkyl;

R₉ is hydrogen or a C₁₋₄ alkyl;

R₁₀ is C₁₋₁₀ alkyl C(O)₂R₈;

R₁₁ is selected from the group consisting of hydrogen, optionally substituted C₁₋₄ alkyl, optionally substituted aryl, optionally substituted aryl C₁₋₄ alkyl, optionally substituted heteroaryl, optionally substituted heteroaryl C₁₋₄ alkyl, optionally substituted heterocyclic, and optionally substituted heterocyclic C₁₋₄ alkyl;

R₁₃ is selected from the group consisting of C₁₋₄ alkyl, aryl, aryl C₁₋₄ alkyl, heteroaryl, heteroaryl C₁₋₄ alkyl, heterocyclic, and heterocyclic C₁₋₄ alkyl;

m is an integer having a value of 0 to 4;

m′ is 0, or an integer having a value of 1 or 2;

q is 0, or an integer having a value of 1 to 10;

s is an integer having a value of 1 to 3; and

t is 0, or an integer having a value of 1 or 2.

Preferred compounds of the present invention are of the formula (II):

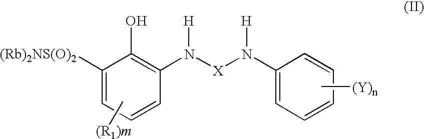

wherein:

R_{b} is independently selected from the group consisting of hydrogen, NR₆R₇, OH, ORₐ, C₁₋₅alkyl, aryl, arylC₁₋₄alkyl, aryl C₂₋₄alkenyl, cycloalkyl, cycloalkyl C₁₋₅ alkyl, heteroaryl, heteroarylC₁₋₄alkyl, heteroarylC₂₋₄ alkenyl, heterocyclic, heterocyclic C₁₋₄alkyl, and a heterocyclic C₂₋₄alkenyl moiety, all of which moieties may be optionally substituted one to three times independently by a substituent selected from the group consisting of halogen, nitro, halosubstituted C₁₋₄ alkyl, C₁₋₄ alkyl, amino, mono or di-C₁₋₄ alkyl substituted amine, ORₐ, C(O)Rₐ, NRₐC(O)ORₐ, OC(O)NR₆R₇, hydroxy, NR₉C(O)Rₐ, S(O)ₘ′Rₐ, C(O)NR₆R₇, C(O)OH, C(O)ORₐ, S(O)₂NR₆R₇, and NHS(O)₂Rₐ, or the two R_{b} substituents can join to form a 3-10 membered ring, optionally substituted and containing, in addition to carbon, independently, 1 to 3 substituents selected from the group consisting of NRₐ, O, S, SO, and SO₂, which substituents can be optionally unsaturated;

Rₐ is selected from a group consisting of alkyl, aryl, arylC₁₋₄alkyl, heteroaryl, heteroaryl C₁₋₄alkyl, heterocyclic, COORₐ, and a heterocyclic C₁₋₄alkyl moiety, all of which moieties may be optionally substituted;

m is an integer having a value of 0 to 3;

m′ is 0, or an integer having a value of 1 or 2;

n is an integer having a value of 0 to 5;

q is 0, or an integer having a value of 1 to 10;

t is 0, or an integer having a value of 1 or 2;

s is an integer having a value of 1 to 3;

R₁ is independently selected from the group consisting of hydrogen, halogen, nitro, cyano,C₁₋₁₀ alkyl, halosubstituted C₁₋₁₀ alkyl, C₂₋₁₀ alkenyl, C₁₋₁₀ alkoxy, halosubstituted C₁₋₁₀alkoxy, azide, S(O)ₜR₄, (CR₈R₈)_{q} S(O)ₜR₄, hydroxy, hydroxy substituted C₁₋₄alkyl, aryl, aryl C₁₋₄ alkyl, aryl C₂₋₁₀ alkenyl, aryloxy, aryl C₁₋₄ alkyloxy, heteroaryl, heteroarylalkyl, heteroaryl C₂₋₁₀ alkenyl, heteroaryl C₁₋₄ alkyloxy, heterocyclic, heterocyclic C₁₋₄alkyl, heterocyclicC₁₋₄alkyloxy, heterocyclicC₂₋₁₀ alkenyl, (CR₈R₈)_{q} NR₄R₅, (CR₈R₈)_{q} C(O)NR₄R₅, C₂₋₁₀ alkenyl C(O)NR₄R₅, (CR₈R₈)_{q} C(O)NR₄R₁₀, S(O)₃R₈, (CR₈R₈)_{q} C(O)R₁₁, C₂₋₁₀ alkenyl C(O)R₁₁, C₂₋₁₀ alkenyl C(O)OR₁₁, (CR₈R₈)_{q} C(O)OR₁₁, (CR₈R₈)_{q} OC(O)R₁₁, (CR₈R₈)_{q}NR₄C(O)R₁₁, (CR₈R₈)_{q} C(NR₄)NR₄R₅, (CR₈R₈)_{q NR}₄C(NR₅) R₁₁, CR₈R₈)_{q} NHS(O)₂R₁₃, and (CR₈R₈)_{q} S(O)₂NR₄R₅, or two R₁ moieties together may form O—(CH₂)ₛO or a 5 to 6 membered saturated or unsaturated ring, wherein the alkyl, aryl, arylalkyl, heteroaryl, heterocyclic moieties may be optionally substituted;

R₄ and R₅ are independently selected from the group consisting of hydrogen, optionally substituted C₁₋₄ alkyl, optionally substituted aryl, optionally substituted aryl C₁₋₄alkyl, optionally substituted heteroaryl, optionally substituted heteroaryl C₁₋₄alkyl, heterocyclic, and heterocyclicC₁₋₄ alkyl; or R₄ and R₅ together with the nitrogen to which they are attached form a 5 to 7 member ring which may optionally comprise an additional heteroatom selected from oxygen, nitrogen and sulfur;

R₆ and R₇ are independently selected from the group consisting of hydrogen, C₁₋₄ alkyl, heteroaryl, aryl, aklyl aryl, and alkyl C₁₋₄ heteroalkyl; or R₆ and R₇ together with the nitrogen to which they are attached form a 5 to 7 member ring which ring may optionally contain an additional heteroatom is selected from oxygen, nitrogen or sulfur, and which ring may be optionally substituted;

Y is selected from the group consisting of hydrogen, halogen, nitro, cyano, halosubstituted C₁₋₁₀ alkyl, C₁₋₁₀ alkyl, C₂₋₁₀ alkenyl, C₁₋₁₀ alkoxy, halosubstituted C₁₋₁₀ alkoxy, azide, (CR₈R₈)_{q}S(O)ₜRₐ, (CR₈R₈)_{q}ORₐ, hydroxy, hydroxy substituted C₁₋₄alkyl, aryl, aryl C₁₋₄ alkyl, aryloxy, arylC₁₋₄ alkyloxy, aryl C₂₋₁₀ alkenyl, heteroaryl, heteroarylalkyl, heteroaryl C₁₋₄ alkyloxy, heteroaryl C₂₋₁₀ alkenyl, heterocyclic, heterocyclic C₁₋₄alkyl, heterocyclicC₂₋₁₀ alkenyl, CR₈R₈)_{q}NR₄R₅, C₂₋₁₀ alkenyl C(O)NR₄R₅, (CR₈R₈)_{q}C(O)NR₄R₅, (CR₈R₈)_{q} C(O)NR₄R₁₀, S(O)₃R₈, (CR₈R₈)_{q}C(O)R₁₁, C₂₋₁₀ alkenylC(O)R₁₁, (CR₈R₈)_{q}C(O)OR₁₁, C₂₋₁₀alkenylC(O)OR₁₁, (CR₈R₈)_{q}OC(O)R₁₁, (CR₈R₈)_{q}NR₄C(O)R₁₁, (CR₈R₈)_{q} NHS(O)₂R₁₃, (CR₈R₈)_{q} S(O)₂NR₄R₅, (CR₈R₈)_{q}C(NR₄)NR₄R₅, and (CR₈R₈)_{q} NR₄C(NR₅)R₁₁; or two Y moieties together may form O—(CH₂)ₛ—O or a 5 to 6 membered saturated or unsaturated ring wherein the alkyl, aryl, arylalkyl, heteroaryl, heteroaryl alkyl, heterocyclic, heterocyclicalkyl groups may be optionally substituted;

R₈ is hydrogen or C₁₋₄ alkyl;

R₉ is hydrogen or a C₁₋₄ alkyl;

R₁₀ is C₁₋₁₀ alkyl C(O)₂R_{8;}

R₁₁ is selected from the group consisting of hydrogen, optionally substituted C₁₋₄ alkyl, optionally substituted aryl, optionally substituted aryl C₁₋₄alkyl, optionally substituted heteroaryl, optionally substituted heteroarylC₁₋₄alkyl, optionally substituted heterocyclic, and optionally substituted heterocyclicC₁₋₄alkyl;

R₁₃ is selected from the group consisting of C₁₋₄ alkyl, aryl, aryl C₁₋₄alkyl, heteroaryl, heteroarylC₁₋₄alkyl, heterocyclic, or heterocyclicC₁₋₄alkyl; and

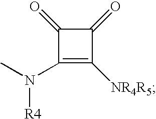

and

X is C=O;

or a pharmaceutically acceptable salt thereof.

Illustrative compounds of Formula (I) and (II) include, but are not limited to:

N-(2-Hydroxyl-3-aminosulfonyl4-chlorophenyl)-N′-(2-bromophenyl) urea;

N-(2-Hydroxy-3-aminosulfonyl-4-chlorophenyl)-N′-(2,3-dichlorophenyl) urea;

N-[2-Hydroxy-3-(N″,N″-dimethyl)-aminosulfonyl-4-chlorophenyl]-N′-(2,3-dichlorophenyl) urea;

N-(2-Hydroxy-3-N″,N″-dimethylaminosulfonyl-4-chlorophenyl)-N′-(2-bromophenyl) urea;

N-(2-Hydroxy-3-N″-methylaminosulfonyl-4-chlorophenyl)-N′-(2-bromophenyl) urea;

N-(2-Hydroxy-3-N″-methylaminosulfonyl-4-chlorophenyl)-N′-(2,3-dichlorophenyl) urea;

N-[⁴-chloro-2-hydroxy-3-[N″-(2-methoxyethyl)aminosulfonyl]phenyl]-N′-(2,3-dichlorophenyl) urea

1-(4-Chloro-2-hydroxy-3-methanesulfonyl-phenyl)-3-(2,3-dichloro-phenyl)-urea;

1-(2-Bromo-phenyl)-3-(4-cyano-2-hydroxy-3-methanesulfonyl-phenyl)-urea;

1-(2-Bromo-phenyl)-3-(4-cyano-2-hydroxy-3-propyl-phenyl)-urea;

1-(2-Bromo-phenyl)-3-[4-cyano-2-hydroxy-3-(1-methyl-butyl)-phenyl]-urea;

1-(2-Bromo-phenyl)-3-(4-cyano-2-hydroxy-3-isobutyl-phenyl)-urea;

1-(3-Bromo-4-cyano-2-hydroxy-phenyl)-3-(2-bromo-phenyl)-urea;

1-(4Chloro-2-hydroxy-3-methanesulfinyl-phenyl)-3-(2,3-dichloro-phenyl)-urea;

{6-Chloro-3-[3-(2,3-dichloro-phenyl)-ureido]-2-hydroxy-phenyl }-methanesulfonamide;

3-[3-(2-Bromo-phenyl)-ureido]-6-chloro-2-hydroxy-benzamide;

6-Chloro-3-[3-(2,3-dichloro-phenyl)-ureido]-2-hydroxy-N-phenyl-benzamide;

1-[4-Chloro-2-hydroxy-3-(1-morpholin-4-yl-methanoyl)-phenyl]-3-(2,3-dichloro-phenyl)-urea;

6-Chloro-3-(3,4-dioxo-2-phenylamino-cyclobut-1 -enylamino)-2-hydroxy-benzenesulfonamide;

3-(3,4-Dioxo-2-phenylamino-cyclobut- 1-enylamino)-2-hydroxy-benzonitrile;

3-(3-Fluoro-2-hydroxy-phenylamino)-4-phenylamino-cyclobut-3-ene-1,2-dione;

4-(3,4-Dioxo-2-phenylamino-cyclobut-l-enylamino)-3-hydroxy-benzonitrile; and

3-(2-Hydroxy4-nitro-phenylamino)-4-phenylamino-cyclobut-3-ene-1,2-dione.

Preferred compounds in the present invention have a half life of 2 hours or above, more preferably 5 hours or above, even more preferably 10 hours or above. Preferred compounds of the present invention exhibit a clearance value Clᵢₙₜ of one or below, more preferably 0.8 or below, even more preferably 0.6 or below. Preferred compounds of the present invention maintain the acidity of the phenol moiety, exhibiting a pKa of 8.5 or below, more preferably a pKa of 8.0 or below, even more preferably 7.0 or below.

### EXPERIMENTAL RESULTS

In contrast to the findings of Temellini, the present invention discloses that the introduction of a sulfonamide or sulfoxide group ortho to the phenol reduced the rate of conjugation of the phenol and hence increased the half-life of the compounds in vivo. Other functional groups were less effective in blocking glucuronidation of the phenol. For example, a series of IL-8 inhibitors containing a sulfonamide or sulfone ortho to the phenol were found to have reduced clearance when incubated with UDPGA (Uridinium diphosphate glucuronic acid) in liver microsomes as compared to the corresponding amides, sulfoxides, and alkyl substituted compounds (see Tables 1 and 2).

The standard procedure for these experiments is as follows: incubations were performed in a final volume of 1.0 mL in a heated block at approximately 37° C. Each incubation contained approximately 0.5 mg/mL microsomal protein and 0.5 uM of the compound. The incubations were conducted with 50 mM potassium phosphate buffer (pH 7.4) and, following a 5 min preincubation at 37° C., were initiated by the addition of cofactor (UDPGA, 4 mM). Aliquots were removed every three minutes and were quenched with two volumes of ACN/EtOH/Acetic acid (80:20:1) containing an appropriate internal standard. Samples were store frozen (ca.−70° C.) until analysis for the quantitation of the parent compound by LC/MS methods. The rate of disappearance of each compound was determined from relative concentration versus time profiles fitted to appropriate exponential decay equations. CLᵢₙₜ (mL/min/g liver) was calculated using standard scaling factors.

The data in Table 1 shows that diphenylureas containing a sulfonamide or sulfone ortho to the phenol (entries 1-9) have markedly lower clearance (<0.6 mL/min/g) than compounds containing an alkyl group (entries 10-13, and 16), a halide (entry 14), a sulfoxide (entry 15), or an amide (entries 17-19) ortho to the phenol. Table 2 shows similar data for the squaramide series of compounds.

TABLE 1
Glucoronidation results in rat and human hepatic microsomes.

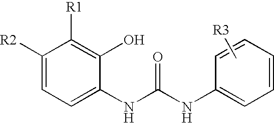

| | | | | Clᵢₙₜ | CLᵢₙₜ |
|---|---|---|---|---|---|
| | | | | (mL/min/g | (mL/min/g |
| | | | | liver) | liver) |
| Entry | R1 | R2 | R3 | rat | human |
| 1 | SO₂NH₂ | Cl | 2-Br | <0.6 | <0.6 |
| 2 | SO₂NH₂ | Cl | 2,3-Cl | 0.64 | <0.6 |
| 3 | SO₂N(Me)₂ | Cl | 2,3-Cl | <0.6 | <0.6 |
| 4 | SO₂N(Me)₂ | Cl | 2-Br | <0.6 | <0.6 |
| 5 | SO₂NHMe | Cl | 2,3-Cl | <0.6 | <0.6 |
| 6 | SO₂NHMe | Cl | 2-Br | <0.6 | <0.6 |
| 7 | SO₂NH(CH₂CH₂OMe) | Cl | 2,3-Cl | <0.6 | <0.6 |
| 8 | SO₂CH₃ | Cl | 2,3-Cl | <0.6 | <0.6 |
| 9 | SO₂CH₃ | CN | 2-Br | <0.6 | <0.6 |
| 10 | propyl | CN | 2-Br | — | 11.3 |
| 11 | C(CH₃)CH₂CH₃ | CN | 2-Br | — | 2.8 |
| 12 | C(CH₃)CH₂CH₂CH₃ | CN | 2-Br | — | 2.4 |
| 13 | CH₂CH(CH₃)₂ | CN | 2-Br | — | 7.6 |
| 14 | Br | CN | 2-Br | 27 | 3.7 |
| 15 | SOCH₃ | Cl | 2,3-Cl | 2.6 | 4.1 |
| 16 | CH₂SO₂NH₂ | Cl | 2,3-Cl | 1.3 | 11 |
| 17 | CONH₂ | Cl | 2-Br | 5.2 | 15.4 |
| 18 | CONHPh | Cl | 2,3-Cl | 9.3 | 11 |
| 19 | 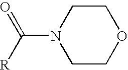 | Cl | 2,3-Cl | 24 | 21 |

TABLE 2
Glucuronidation results in rat and human hepatic microsomes.

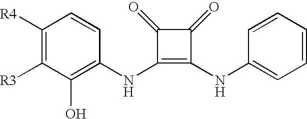

| | | | Clᵢₙₜ (mL/min/g | CLᵢₙₜ (mL/min/g |
|---|---|---|---|---|
| | | | liver) | liver) |
| Entry | R3 | R4 | rat | human |
| 1 | SO₂NH₂ | Cl | <0.6 | <0.6 |
| 2 | CN | H | 0.71 | 0.74 |
| 3 | F | H | 12 | 16 |
| 4 | H | CN | 5.1 | 28 |
| 5 | H | NO₂ | 6.7 | >50 |

Compounds with a sulfonamide ortho to the phenol also showed increased half-life and reduced clearance in vivo as compared to compounds having another function group ortho to the phenol (Table 3)

Method for Determining the in vivo Half-life (T½) of Compounds in Table 3.

The study was conducted using a crossover design on two separate study days. Three male Sprague-Dawley rats received surgically implanted catheters in the vena cava (via the femoral vein), and in the femoral artery at least three days prior to the study. On study day one, the animals (fed) received the compound as a 60 min iv infusion (4.0 mL/kg). The dose solution was prepared in 10% PEG 400 and isotonic saline (pH=3.0 -3.5) and contained 1.4% DMSO. On study day two, the animals (fasted) received the compound by oral gavage (16.0 mL/kg). The dose solution was prepared in 10.0% PEG 400 and water (pH =3.5-4.0) and contained 1.6% DMSO. Blood samples were collected prior to dosing and at various times following administration of compounds. Plasma concentrations of the compounds were quantified by an HPLC/MS/MS method (LLQ=10 ng/mL). Noncompartmental analysis was used for pharmacokinetic analysis of plasma concentration versus time data.

TABLE 3
In vivo half-life and clearance in the rat.

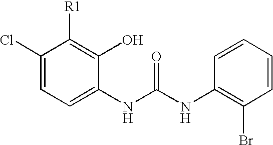

| Entry | R₁ | T_{½}(hrs) | Cl (mL/min/kg) |
|---|---|---|---|
| 1 | SO₂NH₂ | 10.6 | 4.6 |
| 2 | CONH₂ | Nd^{a} | >40 |
| 3 | CONHPh | 0.19 | 65 |
| 4 | S(O)Me | Nd^{a} | 72 |
| 5 | CH₂SO₂NH₂ | 0.09 | 26 |

ND^{a}: The terminal elimation phase was poorly defined, parameter could not be measured.

Method for Determining the pKₐ of a Compound.

The pKₐ of a compound was measured using the following method. The compound (20uM in a 10% DMSO solution) was added to a phosphate buffer solution. The concentrations of compound were then measured using a UV (280 nm) plate reader. Linear regression analysis was then used to determine the pKₐ value as measured by the following equation:

pH=pKₐ+log((Aₘₐₓ−A)/(A−Aₘᵢₙ))

where A is the UV absorbance

Aₘₐₓ is the maximum of abs

Aₘᵢₙ is the minimum of abs.

### METHODS OF PREPARATION

The compounds of Formulas (I) may be obtained by applying synthetic procedures, some of which are illustrated in the Schemes below. The synthesis provided for in these Schemes is applicable for the producing compounds of Formulas (I) having a variety of different R groups which are reacted, employing optional substituents which are suitably protected, to achieve compatibility with the reactions outlined herein. Subsequent deprotection, in those cases, then affords compounds of the nature generally disclosed. Once the urea nucleus has been established, further compounds of these formulas may be prepared by applying standard techniques for functional group interconversion, well known in the art.

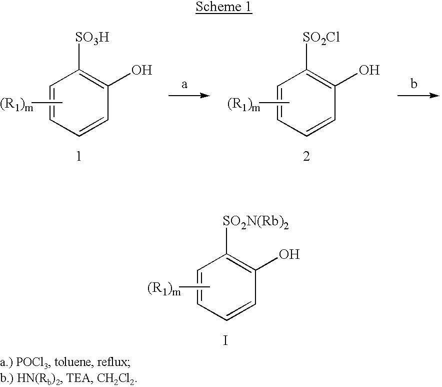

The desired compounds of formula (I) can be obtained from commercially available sulfonic acids 1 as outlined in scheme 1. The sulfonic acid 1 can be converted to the sulfuryl chloride 2 using methods well known in the art such as phosphorous oxychloride in refluxing toluene. The sulfuryl chloride 2 can be coupled with the desired amine (HN(R_{b})₂) to give the sulfonamide (I) using standard techniques well known in the art such as the desired amine in a suitable organic solvent such as methylene chloride in the presence of an amine base such as triethylamine.

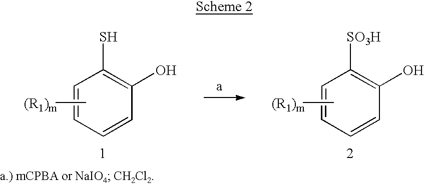

If the desired sulfonic acid 1 is not commercially available, it can be prepared from a commercially available thiol as outlined in scheme 2. The sulfonic acid 2 can be prepared from the thiol 1 using oxidizing conditions well know in the art such as meta-chlorobenzoic acid (mCPBA) or sodium periodate (NaIO₄) in a suitable organic solvent such as methylene chloride.

If neither the desired sulfonic acid or thiol are commercially available, the desired substituted phenol sulfonamide (I) can be prepared by other methods. The thiol precusor to phenol sulfonamide (I) can be abstained by a nucelophilic displacement reaction as outlined in scheme 3 (Zh. Organ. XIMII 1978, 14, 120(1), 187-192 and J. Med. Chem. 1989, 32, 2396).

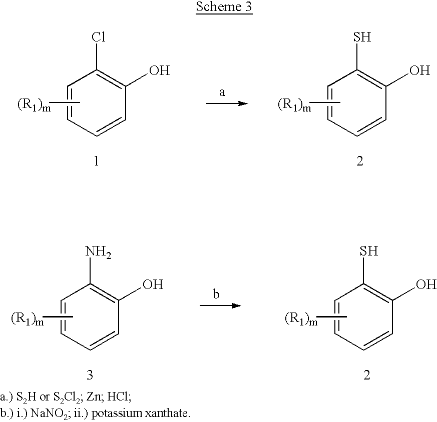

The desired thiol 2 in scheme 3 can be obtained from a commercially available ortho chloro phenol 1 or ortho amino phenol 3 as outlined in scheme 3. The ortho chloro phenol can be reacted with hydrogen sulfide or dichlorosulfide in the presence of zinc and hydrochloric acid to give the desired thiol 2. The ortho amino phenol 3 can be converted to the thiol 2 via the intermediate azide (not shown). The azide can be obtained from the aniline 3 using conditions well known in the art such as sodium nitrate (NaNO₃) in a suitable organic solvent such as methylene chloride. The azide can be converted to the thiol 2 using potassium xanthate in a suitable organic solvent such as methylene chloride.

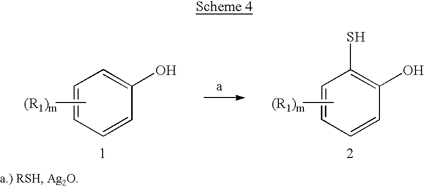

Scheme 4 oultines another method for preparing the desired thiol 2 starting from a commercially available substituted phenol 1 using nucleophilic aromatic substitution chemistry (J. Heterocyclic Chem. 1981, 18(6), 1161-1164). Thus, the thiol group can be introduced by reacting a phenol 1 with the desired thiol (RSH) in the presence of silver oxide (Ag₂O) in a suitable organic solvent such as methylene chloride.

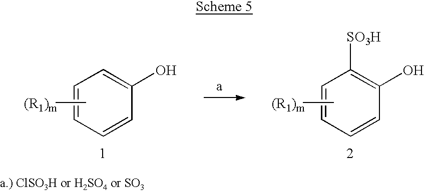

The desired sulfonic acid 2 can also be obtained from a commercially available phenol 1 via electrophilic aromatic substitution chemistry as outlined in scheme 5 (Acta. Chem. Scand. 1979, B33(4), 261-264 and J. Med. Chem. 1981, 24(9), 1063-1067). The phenol 1 can be reacted with either chloro sulfonic acid, sulfuric acid or sulfur trioxide under standard reaction conditions well known in the art to give the sulfonic acid phenol 2.

Compounds of formula (II) can be prepared as outlined below.

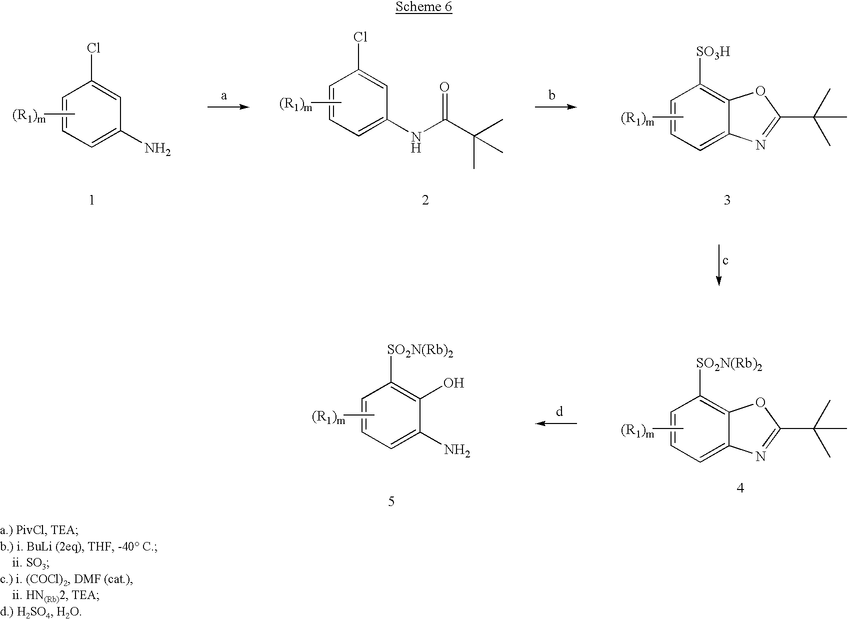

If the desired phenolaniline 5 is not commercially available, it can be prepared as outlined in Scheme 2. Commercially available 3-chloroaniline 1 can be converted to the amide 2 using standard conditions well known in the art such as pivotally chloride and triethylamine in a suitable organic solvent such as methylene chloride. The amide 2 can be converted to the benzoxazole 3 using an excess amount of a strong base such as butyllithium in a suitable organic solvent such as THF under reduced reaction temperatures between −20 and −40° C. followed by quenching the reaction with sulfur trioxide gas. The sulfonic acid 3 can be converted to the sulfonamide 4 using standard conditions well known in the art such as oxalylchloride in a suitable organic solvent such as methylene chloride to give the intermediate sunfonyl chloride. The sulfonyl chloride intermediate can be transformed to the sulfonamide 4 using standard conditions well known in the art by reacting it with the amine HN(R_{b})₂ in the presence of a suitable amine base such as triethylamine in a suitable organic solvent such as methylene chloride. The desired phenolaniline 5 can be obtained from the benzoxazole 4 using standard hydrolysis conditions well known in the art such as sulfuric acid in water and heating at 90° C.

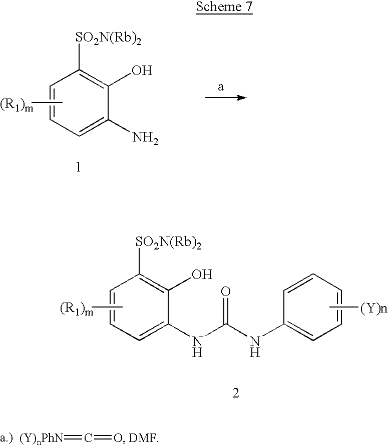

The desired diphenyl ureas 2 can be obtained by condnesing the aniline 1 with the desired isocyanate in a suitable organic solvent such as dimethylformamide (DMF) as outlined in scheme 7. If the desired isocyanate is not commercially available, the isocyanate can be prepared in situ from the aniline using conditions well known in the art such as triphosgene and triethylamine in a suitable organic solvent such as methylene chloride.

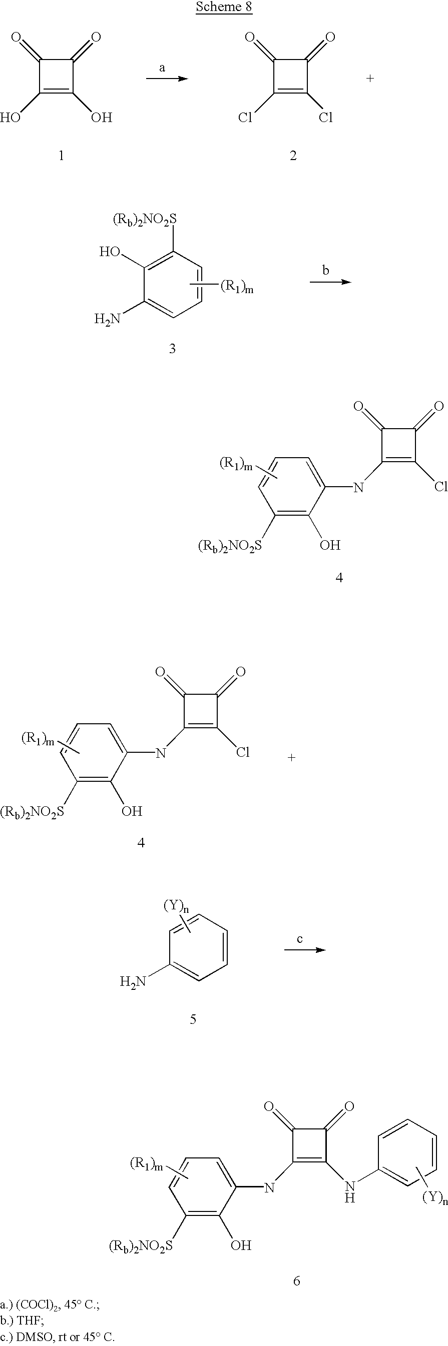

The desired compounds of structure 6 can be prepared as outlined in Scheme 8. Dichlorosquarate 2 can be prepared from squaric acid 1 using standard chlorination methods well known in the art such as oxalyl chloride and catalytic amounts of DMF in methylene chloride and heating at 45° C. Reacting dichlorosquarate 2 with the desired phenolaniline 3 in an organic solvent such as THF gives the mono-chlorosquarate 4. Reacting mono-chlorosquarate 4 with the desired aniline 5 in an organic solvent such as DMSO at room temperature or heating at 45° C. gives the target compound of formula 6.

### EXAMPLES

The invention will now be described by reference to the following examples, which are merely illustrative and are not to be construed as a limitation of the scope of the present invention. All temperatures are given in degrees centigrade, all solvents are highest available purity and all reactions run under anhydrous conditions in an argon atmosphere unless otherwise indicated.

In the Examples, all temperatures are in degrees Centigrade (° C.). Mass spectra were performed upon a VG Zab mass spectrometer using fast atom bombardment, unless otherwise indicated. ¹H-NMR (hereinafter “NMR”) spectra were recorded at 250 MHz using a Bruker AM 250 or Am 400 spectrometer. Multiplicities indicated are: s=singlet, d=doublet, t=triplet, q=quartet, m-multiplet and br indicates a broad signal. Sat. indicates a saturated solution, eq indicates the proportion of a molar equivalent of reagent relative to the principal reactant.

### Example 1

Preparation of N-(4-chloro-2-hydroxy-3-aminosulfonylphenyl)-N′-(2.3-dichlorophenyl) Urea Sodium Salt, and N-(2-bromophenyl)-N′-(4-chloro-2-hydroxy-3-aminosulfonylphenyl) Urea

2,6-Dichlorobenzenesulfonyl chloride

Into a mixture of 200 milliliters (hereinafter “mL”) of acetic acid, water and dichloromethane (3/1/4, v/v/v), 2,6-dichlorobenzenethiol (10.0 grams (hereinafter “g”), 55.8 millimoles (hereinafter “mmol”), N-chlorosuccinimide (37.28 g, 279 mmol) and potassium acetate (2.29 g, 27.9 mmol) were added. The resulting mixture was stirred at 0° C., then warmed to room temperature overnight. The mixture was then diluted with 200 mL of dichloromethane, and washed with water (100 mL×3). The organic layer was dried (Na₂SO₄) and concentrated to give the desired product (11 g, 80%). ₁H NMR (CDCl₃): δ 7.57 (d, 2H), 7.47 (t, 1H).

2,6Dichlorobenzenesulfonamide

A solution of 2,6-dichlorobenzenesulfonyl chloride (10.50 g, 42.77 mmol) in 100 mL of pyridine was added dropwise to 100 mL of pyridine while anhydrous ammonia gas was bubbled through the solution. After 4 hours at 0° C., the mixture was acidified to pH>1 with 6N aq. HCI, then extracted with ethyl acetate. The combined organic layer was then dried (Na₂SO₄) and concentrated to give the desired product (8.69 g, 90%). EI-MS (m/z) 225.0, 227.1 (M⁻).

2,6-Dichloro-3-nitrobenzenesulfonamide

Into a solution of 2,6-dichlorobenzenesulfonamide (7.8 g, 34.5 mmol) in 30 mL of concentrated sulfuric acid at 0°, nitric acid (1.74 mL, 41.4 mmol) was added dropwise. The mixture was stirred at 0° C. for 2 hours, then 200 mL of water was added to produce a precipitate. The resulting mixture was filtered. The white solid was collected, washed with water and dried in vacuo to give the desired product (7.17 g, 76%). ¹H NMR (DMSO-d₆): δ 8.25 (s, 2H), 8.20 (d, 1H), 7.92 (d, 1H).

2-Acetyl-6-chloro-3-nitrobenzenesulfonamide

A solution of 2,6-dichloro-3-nitrobenzenesulfonamide (2.04 g, 7.5 mmol), potassium acetate (2.21 g, 22.5 mmol) and 18-crown-6 (5.95 g, 22.5 mmol) in 50 mL of dimethyl sulfoxide was heated to 45° C. for 7 days. The mixture was acidified with 1N aq. HCl, and extracted with ethyl acetate. The organic layer was concentrated to give the crude material. Column chromatography on silica gel, eluting with ethyl acetate/hexanelacetic acid (50/49/1, v/v/v) gave the desired product (1.67 g, 76%). EI-MS (m/z) 293.1, 295.1 (M⁻)

6-Chloro-2-hydroxy-3-nitrobenzenesulfonamide

A solution of 2-acetyl-6-chloro-3-nitrobenzenesulfonamide (1.72 g, 5.83 mmol), chlorotrimethylsilane (2 mL) and fuming sulfuric acid (0.5 mL) in methanol was heated to reflux for 20 hours. The solvent was evaporated. The residue was diluted with ethyl acetate and washed with water. The organic layer was then dried (Na₂SO₄) and concentrated to give the desired product (1.0 g, 68%). EI-MS (m/z) 251.1, 253.2 (M⁻).

3-Amino-6-chloro-2-hydroxybenzenesulfonamide

To a solution of 6-chloro-2-hydroxy-3-nitrobenzenesulfonamide (1.1 g, 4.36 mmol) in ethyl acetate, was added 10 % Pd/C (500 mg). The mixture was flushed with argon, and then stirred under a hydrogen atmosphere at balloon pressure for 4 hours at room temperature. The mixture was filtered through celite and the celite was washed with methanol. The solvent was evaporated to give the desired product (0.9g, 93%). El-MS (m/z) 221.1, 223.1 (M⁻).

N-(4-Chloro-2hydroxy-3-aminosulfonylphenyl)-N′-(2,3-dichlorophenyl) urea

A solution of 3-amino-6-chloro-2-hydroxybenzenesulfonamide (0.88 g, 3.9 mmol) and 2,3-dichlorophenylisocyanate (0.62 mL, 4.6 mmol) in 5 mL of N,N-dimethyl-formamide was stirred at room temperature for 20 hours. The mixture was diluted with ethyl acetate and washed with water to give the crude material. Purification by column chromatography on silica gel, eluting with ethyl acetate/hexane (30/70 to 50/50, v/v), followed by recrystallization from dichloromethane and hexane, gave the desired product (1.18 g, 74%). mp 241-242° C.

N-(2-Bromophenyl)-N′-(4-chloro-2-hydroxy-3-aminosulfonylphenyl) urea

A solution of 3-amino-6-chloro-2-hydroxybenzenesulfonamide (65 mg, 0.29 mmol) and 2,3dichlorophenylisocyanate (45 μL, 0.36 mmol) in 2 mL of N,N-dimethyl-formamide was stirred at room temperature for 20 hours. The mixture was diluted with ethyl acetate and washed with water to give the crude material. Purification by column chromatography on silica gel, eluting with ethyl acetate/hexane (30/70 to 40/60, v/v), gave the desired product (50 mg, 41%). EI-MS (m/z) 418.2, 420.2, 422.2 (M⁻).

N-(4-chloro-2-hydroxy-3-aminosulfonylphenyl)-N′-(2,3-dichlorophenyl) urea sodium salt

To a solution of N-(4-chloro-2-hydroxy-3-aminosulfonylphenyl)-N′-(2,3-dichlorophenyl) urea (1.47 g, 59 mmol) in 150 mL of acetone was added 2.46 mL of aq. NaOH solution (1.45 M). The mixture was stirred for 16 hours at room temperature and the solvent was evaporated. The residue was recrystallized from acetone and dichloromethane to give the desired product (1.41 g, 91%). ₁H NMR (DMSO-d₆): δ 9.27 (s, 2H), 8.01 (m, 3H), 7.77 (d, 1H), 7.26 (m, 2H), 6.05 (d, 11H)

### Examples 3 & 4

Preparation of N-[4-chloro-3-(N″,N″-dimethylaminosulfonyl)-2-hydroxyphenyl]-N′-(2,3-dichlorophenyl) urea and N-(2-bromophenyl)-N′-[4-chloro-3-(N″,N″-dimethylaminosulfonyl)-2-hydroxyphenyl] urea

N,N-dimethyl-6-chloro-2-hydroxy-3-nitrobenzenesulfonamide

To a mixture of 2-acetyl-6-chloro-3-nitrobenzenesulfonamide (300 mg, 1.02 mmol) and sodium hydride (122 mg, 3.06 mmol) in 10 mL of N,N-dimethylformamide, was added iodomethane (0.64 mL, 10.2 mmol). The mixture was stirred at room temperature for 20 hours. The resulting mixture was acidified with 1N aq. HCl, then extracted with ethyl acetate. The solvent was concentrated to give the crude material. Column chromatography on silica gel, eluting with ethyl acetate/hexane/acetic acid (50/49/1, v/v/v), gave the desired product (140 mg, 49%). ¹H NMR (DMSO-d₆): δ 8.05 (d, 1H), 7.03 (d, 1H), 2.87 (s, 6H).

N,N-Dimethyl-3-amino-6-chloro-2-hydroxybenzenesulfonamide.

To a solution of N,N-dimethyl-6-chloro-2-hydroxy-3-nitrobenzenesulfonamide (140 mg, 0.50 mmol) in ethyl acetate, was added 10% Pd/C (50 mg). The mixture was flushed with hydrogen, then stirred under a hydrogen atmosphere at balloon pressure for 1.5 hours at room temperature. The mixture was filtered through celite and the celite was washed with methanol. The solvent was evaporated to give the desired product (100 mg, 80%). ₁H NMR (DMSO-d₆): δ 6.87 (d, 1H), 6.80 (d, 1H), 2.82 (s, 6H).

N-[4-Chloro-3-(N″,N″-dimethylaminosulfonyl)-2-hydroxyphenyl]-N′-(2,3-dichlorophenyl) urea.

A solution of N,N-dimethyl-3-amino-6-chloro-2-hydroxybenzenesul formamide (80 mg, 0.32 mmol) and 2,3-dichlorophenylisocyanate (50 μL, 0.38 mmol) in 2 mL of N,N-dimethylformamide was stirred at room temperature for 20 hours,. The mixture was diluted with ethyl acetate and washed with water to give the crude material. Purification by-column chromatography on silica gel, eluting with ethyl acetate/hexane (20/80, v/v), followed by recrystallization from ethyl acetate and hexane, gave the desired product (63 mg, 45%). ₁H NMR (DMSO-d₆): δ 10.51 (s, 1H), 9.34 (s, 1H), 9.27 (s, 1H), 8.29 (d, 1H), 7.32 (m, 2H), 7.16 (d, 1H), 2.87 (s, 6H).

N-(2-Bromophenyl)-N′[4-chloro-3-(N″,N″-dimethylaminosulfonyl)-2-hydroxyphenyl] urea.

A solution of N,N-dimethyl-3-amino-6-chloro-2-hydroxy benzenesul-formamide (80 mg, 0.32 mmol) and 2-bromophenylisocyanate (47 μL, 0.38 mmol) in 2 mL of N,N-dimethylformamide was stirred at room temperature for 20 hours. The mixture was diluted with ethyl acetate and washed with water to give the crude material. Purification by column chromatography on silica gel, eluting with ethyl acetate/hexane (20/80, v/v), followed by recrystallization from ethyl acetate and hexane, gave the desired product (88 mg, 62%). EI-MS (m/z) 446.2,448.3, 450.3 (M⁻).

### Examples 5 & 6

Preparation of N-[4-chloro-2-hydroxy-3-(N″-methylaminosulfonyl)phenyl]-N′-(2,3-dichlorophenyl) urea and N-(2-bromophenyl)-N′-[4-chloro-2-hydroxy-3-(N″-methylaminosulfonyl)phenyl] urea

N-Methyl-2-acetyl-6-chloro-3-nitrobenzenesulfonamide.

To a mixture of 2-acetyl-6-chloro-3-nitrobenzenesulfonamide (300 mg, 1.02 mmol) and sodium hydride (53 mg, 1.32 mmol) in 10 mL of N,N-dimethylformamide, iodomethane (70 μL, 1.12 mmol) was added. The mixture was stirred at room temperature for 66 hours. The mixture was acidified with 1N aq. HCl, then extracted with ethyl acetate. The solvent was concentrated to give the crude material. Column chromatography on silica gel, eluting with ethyl acetate/hexane/acetic acid (50/49/1, v/v/v), gave the desired product (185 mg, 59%). EI-MS (m/z) 307.3, 369.3 (M⁻).

N-Methyl-6-chloro-2-hydroxy-3-nitrobenzenesulfonamide.

A solution of N-methyl-2-acetyl-6-chloro-3-nitrobenzenesulfonamide (170 mg, 0.55 mmol), 0.5 mL of chlorotrimethylsilane and 3 drops of fuming sulfuric acid in ethanol was heated to reflux for 20 hours. The solvent was evaporated. The residue was diluted with ethyl acetate and washed with water. The organic layer was then dried (Na₂SO₄) and concentrated to give the desired product (160 mg, >100%). EI-MS (m/z) 265.2, 267.2 (M⁻).

N-Methyl-3-amino-6-chloro-2-hydroxybenzenesulfonamide.

To a solution of N-methyl-6-chloro-2-hydroxy-3-nitrobenzenesulfonamide (140 mg, 0.53 mmol) in ethyl acetate, was added 10% Pd/C (60 mg). The mixture was flushed with argon, then stirred under a hydrogen atmosphere at balloon pressure for 1.5 hours at room temperature. The mixture was filtered through celite and the celite was washed with methanol. The solvent was evaporated to give the desired product (160 mg, >100%). ¹H NMR (DMSO-d₆): δ 8 7.95 (bs, 1H), 6.85 (d, 1H), 6.79 (d, 1H), 2.48 (d, 3H).

N-[4-chloro-2-hydroxy-3-(N″-methylaminosulfonyl)phenyl]-N′-(2,3-dichlorophenyl) urea

A solution of N-methyl-3-amino-6-chloro-2-hydroxybenzenesulfonamide (70 mg, 0.29 mmol) and 2,3-dichlorophenylisocyanate (57 μL, 0.44 mmol) in 2 mL of N,N-dimethylformamide was stirred at room temperature for 66 hours. The mixture was diluted with ethyl acetate and washed with water to give the crude material. Purification by column chromatography on silica gel, eluting with ethyl acetate/hexane (30/70, v/v), gave the desired product (60 mg, 49%, three steps). EI-MS (m/z) 422.3, 424.3, 426.3 (M⁻).

N′-(2-bromophenyl)-N′-[4-chloro-2-hydroxy-3-(N″-methylaminosulfonyl)phenyl] urea

A solution of N-methyl-3-amino-6-chloro-2-hydroxybenzenesulfonamide (70 mg, 0.29 mmol) and 2-bromophenylisocynate (55 μL, 0.44 mmol) in 2 mL of N,N-dimethylformamide was stirred at room temperature for 66 hours. The mixture was diluted with ethyl acetate and washed with water to give the crude material. Purification by column chromatography on silica gel, eluting with ethyl acetate/hexane (30/70, v/v), gave the desired product (85 mg, 67%, three steps). EI-MS (m/z) 432.2, 434.2, 436.3 (M⁻).

Using analogous methods to those indicated in examples 5 and 6 the following additional compounds were prepared:

### Example 7

N-[4-chloro-2-hydroxy-3-[N″-(2-methoxyethyl)sufonyl]phenyl]-N′-(2,3-dichlorophenyl) urea

The procedure outlined in examples 5 and 6 was followed to give N-[4-chloro-2-hydroxy-3-[N″-(2-methoxyethyl)sufonyl]phenyl]-N′-(2,3-dichlorophenyl)urea; Element Analysis Theory: C 41.00%, H 3.44%, N 8.96%, Found: C 40.77%, H 3.28%, N 8.83%.

### Example 8

1-(4Chloro-2-hydroxy-3-methanesulfonyl-phenyl)-3-(2,3-dichloro-phenyl)-urea

Using the procedure outlined in examples 5 and 6, 1-(4-Chloro-2-hydroxy-3-methanesulfonyl-phenyl)-3-(2,3-dichloro-phenyl)-urea was prepared. LCMS (m/z) 411 (M⁺).

### Example 9

1-(2-Bromo-phenol)-3-(4-cyano-2-hydroxy-3-methanesulfonyl-phenyl)-urea

Using the procedure outlined in examples 5 and 6, 1-(2-Bromo-phenyl)-3-(4-cyano-2-hydroxy-3-methanesulfonyl-phenyl)-urea was prepared. LCMS 412 (m/z) (M⁺).

### Example 10

Standard Procedure for the Synthesis of Alkyl-substituted Phenolic Ureas Synthesis of 1-(2-Bromo-phenyl)-3-(4-cyano-2-hydroxy-3-propyl-phenyl)-urea

2-allyloxy4-cyanonitrobenzene.

To a solution of 2-nitro-5-cyanophenol(1.03g, 6.29 mmol) in dry DMF (10 mL) was added cesium carbonate(2.19 g, 6.7 1 mmol), and the reaction was stirred at 25° C. for 16 h under Ar. The reaction was diluted with EtOAc, washed with satd NaHCO₃, dried MgSO₄, and concentrated to give the title compound(1.21 g, 95%). ¹H NMR (CDCl₃) d 7.88 (d, 1H, J=8.07 Hz), 7.37 (s, 1H), 7.35 (d, 1H, J=7.97 Hz) 6.04 (m, 1H), 5.51 (dd, 1H, J=17.11 Hz). 1.20 Hz), 5.41 (dd, 1H, J=9.42 Hz, 1.16 Hz), 4.74 (d, 2H, J=6.58 Hz).

2-allyloxy-4-cyanoaniline.

To a solution of aniline (9.60 mmol) in ethanol (100 mL) was added SnCI₂ (28.85 mmol). The reaction was stirred at 70 ° C. for 4 h. The reaction mixture was poured into ice, pH was adjusted to 7 with sodium bicarbonate, and extracted with ethyl acetate. The organic layer was dried with MgSO₄, filtered, and concentrated. Flash chromatography (2% MeOH/CH₂Cl₂) afforded 2-allyloxy-4-cyanonitrobenzene (96%). ¹H NMR (CDCl₃) d 7.12 (d, 1H, J=8.05 Hz), 6.98 (s, ¹H), 6.68 (d, 1H, J=8.12 Hz), 6.05 (m, 1H), 5.40 (m, 2H), 4.59 (d, 2H, J=6.13 Hz), 4.31 (bs, 2H); EI-MS nm/z 175(M+H)⁺.

4-cyan o-2-hydroxy-3-(2-propene)aniline 2-allyloxy-4-cyanoaniline(1.49 g, 8.55 mmol) was dissolved in dimethylaniline(15 mL).

The solution was heated under Ar at 175° C. for 3 h. The solution was cooled and then purified directly on silica gel (70% Hexane/30%EtOAc) to give the title compound(1.33 g, 89%). ¹H NMR (CDCl₃) d 7.12 (d, 1H, J=8.10 Hz), 6.62 (d, 1H, J=8.19 Hz) 6.01 (m, 1H), 5.28 (m, 3H) 4.24 (bs, 2H) 3.63 (d, 2H, J=6.08 Hz); EI-MS m/z 173(M−H)₋.

4cyano-2-hydroxy-3-propylaniline.

A solution of 4-cyano-2-hydroxy-3-(2-propene)anie(0.60 g, 3.44 mmol) in ethyl acetate(25 mL) was flushed with Ar. 10% Pd/C(0.25 g) was added, the mixture was flushed with H₂, and then allowed to stir under hydrogen (balloon pressure) at 25 ° C. for 14 h. The reaction was filtered through celite and concentrated to give the title compound (0.579 g, 95%). ¹H NMR (CDCl₃) d 7.11 (d, 1H, J=8.30 Hz), 6.59 (d, 1H, J=8.35 Hz), 2 J=7.64 Hz), 1.66 (m, 2H), 1.04 (t, 2H, J=7.44 Hz); EI-MS m/z 174.8 (M−H)⁻. 1-(2-Bromo-phenyl)-3-(4-cyano-2-hydroxy-3-propyl-phenyl)-urea.

A solution of 4-cyano-2-hydroxy-3-propylaniline (52.4 mg, 0.297 mmol) in DMF (0.40 mL) was treated with 2-bromophenyl isocyanate (0.297 mmol) for 14 h at 25° C. The product was purified by dilution with methylene chloride and precipitation with hexanes. Filtering afforded the title compound(72 mg, 65%), mp 174-175° C. ¹H NMR (DMSO-d₆) d 9.41 (s, 1H), 9.35 (s, 1H), 9.05 (s, 1H), 8.06 (d, 1H, J=8.55 Hz), 7.90 (d, 1H, J=6.89 Hz), 7.63 (d, 1H, J=7.97 Hz), 7.36 (t, 1H, J=8.36 Hz), 7.27 (d, 1H, J=8.53 Hz), 7.35 (t, 1H, J=7.92 Hz), 2.81 (t, 2H, J=7.41 Hz), 1.58 (q, 2H, J=7.53 Hz), 0.95 (t, 3H, J=7.26 Hz); EI-MS m/z 372(M−H)⁻. Anal. (C₁₇H₁₆BrN₃O₂/1H₂O) C,HN: calcd, 52.06, 4.63, 10.71; found, 51.71, 4.35, 10.37.

### Example 11

1-(2-Bromo-phenyl)-3-[4-cyano-2-hydroxy-3-(1-methyl-propyl)-phenyl]-urea

The standard procedure outlined in example 10 was followed to give 1-(2-Bromo-phenyl)-3-[4-cyano-2-hydroxy-3-(1-methyl-butyl)-phenyl]-urea. LCMS (m/z) 389 (M₊).

### Example 12

1-(2-Bromo-phenyl)-3-[4-cyano-2-hydroxy-3-(1-methyl-butyl)-phenyl]-urea

The standard procedure outlined in example 10 was followed to give 1-(2-Bromo-phenyl)-3-[4-cyano-2-hydroxy-3-(1-methyl-butyl)-phenyl]-urea. LCMS (m/z) 403 (M⁺).

### Example 13

1-(2-Bromo-phenyl)-3-(4-cyano-2-hydroxy-3-isobutyl-phenyl)-urea

The standard procedure outlined in example 10 was followed to give 1-(2-Bromo-phenyl)-3-[4-cyano-2-hydroxy-3-(1-methyl-butyl)-phenyl]-urea. LCMS (m/z) 389 (M⁺).

### Example 14

1-(3-Bromo-4-cyano-2-hydroxy-phenyl)-3-(2-bromo-phenyl)-urea

Using the procedure outlined in examples 5 and 6, 1-(3-Bromo4-cyano-2-hydroxy-phenyl)-3-(2-bromo-phenyl)-urea was prepared. LCMS (m/z) 413 (M⁺).

### Example 15

1-(4-Chloro-2-hydroxy-3-methanesulfinyl-phenyl)-3-(2,3-dichloro-phenyl)-urea

Using the procedure outlined in examples 5 and 6, 1-(4-Chloro-2-hydroxy-3-methanesulfinyl-phenyl)-3-(2,3-dichloro-phenyl)-urea was prepared. LCMS 395 (m/z) (M⁺).

### Example 16

{6-Chloro-3-[3-(2,3-dichloro-phenyl)-ureido]-2-hydroxy-phenyl}-methanesulfonamide

Using the procedure outlined in examples 5 and 6, {6-Chloro-3-[3-(2,3dichloro-phenyl)-ureido]-2-hydroxy-phenyl }-methane sulfonamide was prepared. LCMS (m/z) 426 (M⁺).

### Example 17

3-[3-(2-Bromo-phenyl)-ureido]-6-chloro-2-hydroxy-benzamide

The standard procedure outlined in example 18 was followed to give 3-[3-(2-Bromo-phenyl)-ureido]-6-chloro-2-hydroxy-benzamide. LCMS (m/z) 385 (M⁺).

### Example 18

Standard Procedure for the Synthesis of 3-amido Phenols. Synthesis of 6-Chloro-3-[3-(2,3-dichloro-phenyl)-ureido]-2-hydroxy-N-phenyl-benzamide

2,6-dichloro-3-nitro-N-phenyl-benzamide:

To a solution of 2,6-dichloro-3-nitrobenzoic acid (499 mg, 2.11 mmol) in methylene chloride (8 ml) at 0° C. was added oxalyl chloride (0.32 ml, 3.67 mmol) and a drop of DMF. The reaction was stirred until bubbling ceased and then was warmed to room temperature and concentrated. The crude reaction mixture was taken up in DMF (5 ml) and chilled to 0° C. Triethylamine (0.32 ml, 2.30 mmol) was added followed by aniline (0.21 ml, 2.30 mmol). The reaction was warmed to room temperature and stirred for 14 h. The reaction was diluted with water, extracted with ethyl acetate, dried MgSO₄, and concentrated. Flash chromatography (70% Hexane/30% EtOAc) on silica gel gave the title compound (406 mg, 62%). ¹H NMR (DMSO-d₆) d 10.91 (bs, 1H), 8.20 (d, 1H, J=8.0) 7.80 (d, 1H, J=8.0) 7.79 (d, 2H, J=8.0) 7.41 (t, 2H) 7.28 (t, 1H); EI-MS m/z 309(M+H)⁻.

6-chloro-2-hydroxy-3-nitro-N-phenylbenzamide:

To a solution of 2,6-dichloro-3-nitro-N-phenyl-benzamide (950 mg, 3.05 mmol) in DMSO (20 ml) was added KOAc (892 mg, 9.09 mmol) and 18-Crown-6 (2.42 g, 9.15 mmol). The reaction was stirred at 101° C. for 23 h. After cooling to room temperature, 10% NaOH was added and the reaction was allowed to stir for 1 h and acidified to pH 1 with 6 N HCI. The reaction was diluted with water, extracted with ethyl acetate, dried MgSO₄, and concentrated. Flash chromatography (70% Hexane/30% EtOAc/0.1% HOAc) on silica gel gave the title compound (392 mg, 44%). ¹H NMR (DMSO-d₆) d 11.13 (bs, 1H), 10.68 (bs, 1H), 8.10 (d, 1H, J=8.5), 7.71 (d, 2H, J=8.5), 7.36 (t, 2H, J=8.4), 7.12 (t, 1H); EI-MS m/z 291(M+H)³¹ .

3-amino-6-chloro-2-hydroxy-N-phenylbenzamide:

The procedure outline in example 10 was followed using 6-chloro-2-hydroxy-3-nitro-N-phenylbenzamide to afford the title compound (94 mg, 86%). ¹H NMR (DMSO-d₆) d 10.35 (bs, 1H), 7.74 (d, 2H, J=8.5), 7.31 (t, 2H), 7.06 (t, 1H) 6.73 (d, 2H, J=8.5); EI-MS n/z 263(M+H)⁺.

6-Chloro-3-[3-(2,3-dichloro-phenyl)-ureido]-2-hydroxy-N-phenyl-benzamide.

A solution of 3-amino-6-chloro-2-hydroxy-N-phenylbenzamide (91.0 mg, 0.346 mmol) in DMF (1.5 ml) was treated with 2,3-dichlorophenyl isocyanate (0.046 ml, 0.348 mmol) for 14 h at room temperature. The reaction mixture was diluted with ethyl acetate and washed with water. The organic layer was dried with MgSO₄, filtered and concentrated. The product was purified by recrystallization using methylene chloride and hexane. Filtering afforded the title compound (63.2 mg, 42%), mp 244-245° C. ¹H NMR (DMSO-d₆) d 10.53 (bs, 1H), 10.00 (s, 1H), 9.25 (s, 1H), 9.14 (s, 1H), 8.11 (d, 1(d, 1H, J=8.7 Hz), 7.75 (d, 2H, J=7.87 Hz), 7.32 (m, 4H), 7.11 (t, 1H), 7.00 (d, 1H, J=8.75); EI-MS m/z 448 (M+H)⁻. Anal. (C₂₀H₁₄N₃O₃Cl₃) C,H,N: calcd, 53.30, 3.13, 9.32; found, 52.94, 2.85, 9.11.

### Example 19

1-[4-Chloro-2-hydroxy-3-(1-morpholin-4-yl-methanoyl)-phenyl]-3-(2,3-dichloro-phenyl)-urea

The standard procedure outlined in example 18 was followed to give 1-[4-Chloro-2-hydroxy-3-(1-morpholin-4-yl-methanoyl)-phenyl]-3-(2,3-dichloro-phenyl)-urea. LCMS (m/z) 445 (M⁺).

### Example 20

6-Chloro-3-(3,4-dioxo-2-phenylamino-cyclobut-1-enylamino)-2-hydroxy-benzenesulfonamide

The following is the standard procedure for the synthesis of dianilino squarates. To a solution of 3-anilino-4-ethoxy-1,2-cyclobut-3-enedione (0.11 g, 0.5 mmol) in toluene (1 mL) was added 3-Amino-6-chloro-2-hydroxy-benzenesulfonamide (0.11 g, 0.5 mmol) and the reaction mixture heated at 110° C. After 24 hrs, the reaction was concentrated and the crude residue purified by titration from acetone/hexanes to give 40 mg (20%) of 6-Chloro-3-(3,4-dioxo-2-phenylamino-cyclobut-1-enylamino)-2-hydroxy-benzenesulfonamide as a tan solid. LCMS (m/z) 394 (M⁺).

### Example 21

3-(3,4-Dioxo-2-phenylamino-cyclobut-1-enylamino)-2-hydroxy-benzonitrile

The standard procedure outlined in example 20 was followed to give 3-(3,4-dioxo-2-phenylamino-cyclobut-1-enylamino)-2-hydroxy-benzonitrile as a tan solid. LCMS (m/z) 306 (M⁺).

### Example 22

3-(3-Fluoro-2-hydroxy-phenylamino)-4-phenylamino-cyclobut-3-ene-1,2-dione

The standard procedure outlined in example 20 was followed to give 3-(3-Fluoro-2-hydroxy-phenylamino)-4-phenylamino-cyclobut-3-ene-1,2-dione as a tan solid. LCMS (m/z) 299 (M⁺).

### Example 23

4-(3,4-Dioxo-2-phenylamino-cyclobut-1-enylamino)-3-hydroxy-benzonitrile

The standard procedure outlined in example 20 was followed to give 4-(3,4-Dioxo-2-phenylamino-cyclobut-1-enylamino)-3-hydroxy-benzonitrile as a tan solid. LCMS (m/z) 306 (M⁺).

Example 24

3-(2-Hydroxy-4-nitro-phenylamino)-4-phenylamino-cyclobut-3-ene-1,2-dione.

The standard procedure outlined in example 20 was followed to give 3-(2-Hydroxy-4-nitro-phenylamino)-4-phenylamino-cyclobut-3-ene-1,2-dione as a tan solid. LCMS (m/z) 326 (M⁺).

All publications, including but not limited to patents and patent applications cited in this specification are herein incorporated by reference as if each individual publication were specifically and individually indicated to be incorporated by reference as though fully set forth.

What is claimed is:

1. A method of increasing the metabolic stability and/or half life of a phenol-containing compound by placing a sufone or sulfonamide substituent ortho to the phenol.

2. A method according to claim 1 wherein the sulfone or sulfonamide moiety has a structure (Rb)₂NS(O)₂ wherein:
R_{b} is independently selected from the group consisting of hydrogen, NR₆R₇, OH, ORₐ, C₁₋₅alkyl, aryl, arylC₁₋₄alkyl, aryl C₂₋₄alkenyl, cycloalkyl, cycloalkyl C₁₋₅ alkyl, heteroaryl, heteroarylC₁₋₄alkyl, heteroarylC₂₋₄ alkenyl, heterocyclic, heterocyclic C₁₋₄alkyl, and a heterocyclic C₂₋₄alkenyl moiety, all of which moieties may be optionally substituted one to three times independently by a substituent selected from the group consisting of halogen, nitro, halosubstituted C₁₋₄ alkyl, C₁₋₄ alkyl, amino, mono or di-C₁₋₄ alkyl substituted amine, ORₐ, C(O)Rₐ, NRₐC(O)ORₐ, OC(O)NR₆R₇, hydroxy, NR₉C(O)Rₐ, S(O)ₘ′Rₐ, C(O)NR₆R₇, C(O)OH, C(O)ORₐ, S(O)₂NR₆R₇, and NHS(O)₂Rₐ, or the two R_{b} substituents can join to form a 3-10 membered ring, optionally substituted and containing, in addition to carbon, independently, 1 to 3 substituents selected from the group consisting of NRₐ, O, S, SO, and SO₂, which substituents can be optionally unsaturated;
Rₐ is selected from a group consisting of alkyl, aryl, arylC₁₋₄alkyl, heteroaryl, heteroaryl C₁₋₄alkyl, heterocyclic, COORₐ, and a heterocyclic C₁₋₄alkyl moiety, all of which moieties may be optionally substituted;
m′ is 0, or an integer having a value of 1 or 2; and R₆ and R₇ are independently selected from the group consisting of hydrogen, C₁₋₄ alkyl, heteroaryl, aryl, aklyl aryl, and alkyl C₁₋₄ heteroalkyl; R₆ and R₇ together with the nitrogen to which they are attached form a 5 to 7 member ring which ring may optionally contain an additional heteroatom is selected from oxygen, nitrogen or sulfur, and which ring may be optionally substituted.

3. A method according to claim 1 wherein the phenol-containing compound is represented by formula (I): 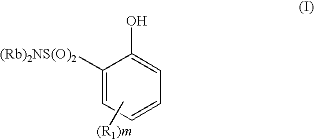
wherein
R_{b} is independently selected from the group consisting of hydrogen, NR₆R₇, OH, ORₐ, C₁₋₅alkyl, aryl, aryl C₁₋₄alkyl, aryl C₂₋₄ alkenyl, cycloalkyl, cycloalkyl C₁₋₅ alkyl, heteroaryl, heteroarylC₁₋₄ alkyl, heteroaryl C₂₋₄ alkenyl, heterocyclic, heterocyclic C₁₋₄ alkyl, and a heterocyclic C₂₋₄ alkenyl moiety, all of which moieties may be optionally substituted one to three times independently by a substituent selected from the group consisting of halogen, nitro, halosubstituted C₁₋₄ alkyl, C₁₋₄ alkyl, amino, mono or di-C₁₋₄ alkyl substituted amine, ORₐ, C(O)Rₐ, NRₐC(O)ORₐ, OC(O)NR₆R₇, hydroxy, NR₉C(O)Rₐ, S(O)ₘ′Rₐ, C(O)NR₆R₇, C(O)OH, C(O)ORₐ, S(O)₂NR₆R₇, and NHS(O)₂Rₐ; or the two R_{b} substituents can join to form a 3-10 membered ring, optionally substituted and containing; in addition to carbon, independently, 1 to 3 substituents selected from the group consisting of NRₐ, O, S, SO, and SO₂, which substituents can be optionally unsaturated;
R₁ is independently selected from the group consisting of hydrogen, halogen, nitro, cyano, C₁₋₁₀ alkyl, halosubstituted C₁₋₁₀ alkyl, C₂₋₁₀ alkenyl, C₁₋₁₀ alkoxy, halosubstituted C₁₋₁₀ alkoxy, azide, S(O)ₜR₄, (CR₈R₈)_{q} S(O)ₜR₄, hydroxy, hydroxy substituted C₁₋₄ alkyl, aryl, aryl C₁₋₄ alkyl, aryl C₂₋₁₀ alkenyl, aryloxy, aryl C₁₋₄ alkyloxy, heteroaryl, heteroaryl alkyl, heteroaryl C₂₋₁₀ alkenyl, heteroaryl C₁₋₄ alkyloxy, heterocyclic, heterocyclic C₁₋₄ alkyl, heterocyclic C₁₋₄ alkyloxy, heterocyclic C₂₋₁₀ alkenyl, NR₄C(O)NR₄R₅, NR₄C(S)NR₄R₅, (CR₈R₈)_{q} NR₄R₅, (CR₈R₈)_{q}C(O)NR₄R₅, C₂₋₁₀ alkenyl C(O)NR₄R₅, (CR₈R₈)_{q} C(O)NR₄R₁₀, S(O)₃R₈, (CR₈R₈)_{q} C(O)R₁₁, C₂₋₁₀ alkenyl C(O)R₁₁, C₂₋₁₀ alkenyl C(O)OR₁₁, (CR₈R₈)_{q} C(O)OR₁₁, (CR₈R₈R₈)_{q} OC(O)R₁₁, (CR₈R₈)_{q}NR₄C(O)R₁₁, (CR₈R₈)_{q} C(NR₄)NR₄R₅, (CR₈R₈)_{q} NR₄C(NR₅)R₁₁, (CR₈R₈)_{q} NHS(O)₂R₁₃, (CR₈R₈)_{q} S(O)₂NR₄R₅, and 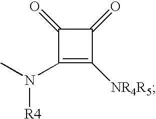
or two R₁ moieties together may form O—(CH₂)ₛO or a 5 to 6 membered saturated or unsaturated ring, and wherein the alkyl, aryl, arylalkyl, heteroaryl, heterocyclic moieties may be optionally substituted;
R₄ and R₅ are independently selected from the group consisting of hydrogen, optionally substituted C₁₋₄ alkyl, optionally substituted aryl, optionally substituted aryl C₁₋₄ alkyl, optionally substituted heteroaryl, optionally substituted heteroaryl C₁₋₄ alkyl, heterocyclic, and heterocyclic C₁₋₄ alkyl; or R₄ and R₅ together with the nitrogen to which they are attached form a 5 to 7 member ring which may optionally comprise an additional heteroatom selected from oxygen, nitrogen and sulfur;
R₆ and R₇ are independently selected from the group consisting of hydrogen, C₁₋₄ alkyl, heteroaryl, aryl, aklylaryl, and alkyl C₁₋₄ heteroalkyl; or R₆ and R₇ together with the nitrogen to which they are attached form a 5 to 7 member ring which ring may optionally contain an additional heteroatom is selected from oxygen, nitrogen or sulfur, and which ring may be optionally substituted;
Rₐ is selected from the group consisting of alkyl, aryl, aryl C₁₋₄ alkyl, heteroaryl, heteroaryl C₁₋₄alkyl, heterocyclic, COORₐ, and a heterocyclic C₁₋₄ alkyl moiety, all of which moieties may be optionally substituted;
R₈ is hydrogen or C₁₋₄ alkyl;
R₉ is hydrogen or a C₁₋₄ alkyl;
R₁₀ is C₁₋₁₀ alkyl C(O)₂R₈;
R₁₁ is selected from the group consisting of hydrogen, optionally substituted C₁₋₄ alkyl, optionally substituted aryl, optionally substituted aryl C₁₋₄ alkyl, optionally substituted heteroaryl, optionally substituted heteroaryl C₁₋₄ alkyl, optionally substituted heterocyclic, and optionally substituted heterocyclic C₁₋₄ alkyl;
R₁₃ is selected from the group consisting of C₁₋₄ alkyl, aryl, aryl C₁₋₄ alkyl, heteroaryl, heteroaryl C₁₋₄ alkyl, heterocyclic, and heterocyclic C₁₋₄ alkyl;
m is an integer having a value of 0 to 4;
m′ is 0, or an integer having a value of 1 or 2;
q is 0, or an integer having a value of 1 to 10;
s is an integer having a value of 1 to 3; and
t is 0, or an integer having a value of 1 or 2.

4. A method according to claim 1 wherein the phenol-containing compound has a half life of 2 hours or above.

5. A method according to claim 1 wherein the phenol-containing compound has a clearance value Clᵢₙₜ of one or below.

6. A method according to claim 1 wherein the phenol-containing compound has a pKa of 8.5 or below.

7. A method according to claim 2 wherein the compound has a structure according to (III): 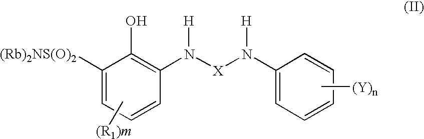
wherein:
R_{b} is independently selected from the group consisting of hydrogen, NR₆R₇, OH, ORₐ, C₁₋₅alkyl, aryl, arylC₁₋₄alkyl, aryl C₂₋₄alkenyl, cycloalkyl, cycloalkyl C₁₋₅ alkyl, heteroaryl, heteroarylC₁₋₄alkyl, heteroarylC₂₋₄ alkenyl, heterocyclic, heterocyclic C₁₋₄alkyl, and a heterocyclic C₂₋₄alkenyl moiety, all of which moieties may be optionally substituted one to three times independently by a substituent selected from the group consisting of halogen, nitro, halosubstituted C₁₋₄ alkyl, C₁₋₄ alkyl, amino, mono or di-C₁₋₄ alkyl substituted amine, ORₐ, C(O)Rₐ, NRₐC(O)ORₐ, OC(O)NR₆R₇, hydroxy, NR₉C(O)Rₐ, S(O)ₘ′Rₐ, C(O)NR₆R₇, C(O)OH, C(O)ORₐ, S(O)₂NR₆R7, and NHS(O)₂Rₐ; or the two R_{b} substituents can join to form a 3-10 membered ring, optionally substituted and containing, in addition to carbon, independently, 1 to 3 substituents selected from the group consisting of NRₐ, O, S, SO, and SO₂, which substituents can be optionally unsaturated;
Rₐ is selected from a group consisting of alkyl, aryl, arylC₁₋₄alkyl, heteroaryl, heteroaryl C₁₋₄alkyl, heterocyclic, COORₐ, and a heterocyclic C₁₋₄alkyl moiety, all of which moieties may be optionally substituted;
m is an integer having a value of 0 to 3;
m′ is 0, or an integer having a value of 1 or 2;
n is an integer having a value of 0 to 5;
q is 0, or an integer having a value of 1 to 10;
t is 0, or an integer having a value of 1 or 2;
s is an integer having a value of 1 to 3;
R₁ is independently selected from the group consisting of hydrogen, halogen, nitro, cyano, C₁₋₁₀ alkyl, halosubstituted C₁₋₁₀ alkyl, C₂₋₁₀ alkenyl, C₁₋₁₀ alkoxy, halosubstituted C₁₋₁₀alkoxy, azide, S(O)ₜR₄, (CR₈R₈)_{q} S(O)ₜR₄, hydroxy, hydroxy substituted C₁₋₄alkyl, aryl, aryl C₁₋₄ alkyl, aryl C₂₋₁₀ alkenyl, aryloxy, aryl C₁₋₄ alkyloxy, heteroaryl, heteroarylalkyl, heteroaryl C₂₋₁₀ alkenyl, heteroaryl, C₁₋₄ alkyloxy, heterocyclic, heterocyclic C₁₋₄alkyl, heterocyclicC₁₋₄alkyloxy, heterocyclicC₂₋₁₀ alkenyl, (CR₈R₈)_{q} NR₄R₅, (CR₈R₈)_{q}C(O)NR₄R₅, C₂₋₁₀ alkenyl C(O)NR₄R₅, (CR₈R₈)_{q} C(O)NR₄R₁₀, S(O)₃R₈, (CR₈R₈)_{q} C(O)R₁₁, C₂₋₁₀ alkenyl C(O)R₁₁, C₂₋₁₀ alkenyl C(O)OR₁₁, (CR₈R₈)_{q} C(O)OR₁₁, (CR₈R₈)_{q} OC(O)R₁₁, (CR₈R₈)_{q}NR₄C(O)R₁₁, (CR₈R₈)_{q} C(NR₄)NR₄R₅, (CR₈R₈)_{q} NR₄C(NR₅)R₁₁, CR₈R₈)_{q} NHS(O)₂R₁₃, and (CR₈R₈)_{q} S(O)₂NR₄R₅; or two R₁ moieties together may form O—(CH₂)ₛO or a 5 to 6 membered saturated or unsaturated ring, wherein the alkyl, aryl, arylalkyl, heteroaryl, heterocyclic moieties may be optionally substituted;
R₄ and R₅ are independently selected from the group consisting of hydrogen, optionally substituted C₁₋₄ alkyl, optionally substituted aryl, optionally substituted aryl C₁₋₄alkyl, optionally substituted heteroaryl, optionally substituted heteroaryl C₁₋₄alkyl, heterocyclic, and heterocyclicC₁₋₄ alkyl; or R₄ and R₅ together with the nitrogen to which they are attached form a 5 to 7 member ring which may optionally comprise an additional heteroatom selected from oxygen, nitrogen and sulfur;
R₆ and R₇ are independently selected from the group consisting of hydrogen, C₁₋₄ alkyl, heteroaryl, aryl, alkyl aryl, and alkyl C₁₋₄ heteroalkyl; R₆ and R₇ together with the nitrogen to which they are attached form a 5 to 7 member ring which ring may optionally contain an additional heteroatom is selected from oxygen, nitrogen or sulfur, and which ring may be optionally substituted;
Y is selected from the group consisting of hydrogen, halogen, nitro, cyano, halosubstituted C₁₋₁₀ alkyl, C₁₋₁₀ alkyl, C₂₋₁₀ alkenyl, C₁₋₁₀ alkoxy, halosubstituted C₁₋₁₀ alkoxy, azide, (CR₈R₈)_{q}S(O)ₜRₐ, (CR₈R₈)_{q}ORₐ, hydroxy, hydroxy substituted C₁₋₄alkyl, aryl, aryl C₁₋₄ alkyl, aryloxy, arylC₁₋₄ alkyloxy, aryl C₂₋₁₀ alkenyl, heteroaryl, heteroarylalkyl, heteroaryl C₁₋₄ alkyloxy, heteroaryl C₂₋₁₀ alkenyl, heterocyclic, heterocyclic C₁₋₄alkyl, heterocyclicC₂₋₁₀ alkenyl, CR₈R₈)_{q}NR₄R₅, C₂₋₁₀ alkenyl C(O)NR₄R₅, (CR₈R₈)_{q}C(O)NR₄R₅, (CR₈R₈)_{q} C(O)NR₄R₁₀, S(O)₃R₈, (CR₈R₈)_{q}C(O)R₁₁, C₂₋₁₀ alkenylC(O)R₁₁, (CR₈R₈)_{q}C(O)OR₁₁, C₂₋₁₀alkenylC(O)OR₁₁, (CR₈R₈)_{q}OC(O)R₁₁, (CR₈R₈)_{q}NR₄C(O)R₁₁, (CR₈R₈)_{q} NHS(O)₂R₁₃, (CR₈R₈)_{q} S(O)₂NR₄R₅, (CR₈R₈)_{q}C(NR₄)NR₄R₅, and (CR₈R₈)_{q} NR₄C(NR₅)R₁₁; or two Y moieties together may form O—(CH₂)ₛ—O or a 5 to 6 membered saturated or unsaturated ring wherein the alkyl, aryl, arylalkyl, heteroaryl, heteroaryl alkyl, heterocyclic, heterocyclicalkyl groups may be optionally substituted;
R₈ is hydrogen or C₁₋₄ alkyl;
R₉ is hydrogen or a C₁₋₄ alkyl;
R₁₀ is C₁₋₁₀ alkyl C(O)₂R₈;
R₁₁ is selected from the group consisting of hydrogen, optionally substituted C₁₋₄ alkyl, optionally substituted aryl, optionally substituted aryl C₁₋₄alkyl, optionally substituted heteroaryl, optionally substituted heteroarylC₁₋₄alkyl, optionally substituted heterocyclic, and optionally substituted heterocyclicC₁₋₄alkyl;
R₁₃ is selected from the group consisting of C₁₋₄ alkyl, aryl, aryl C₁₋₄alkyl, heteroaryl, heteroarylC₁₋₄alkyl, heterocyclic, heterocyclicC₁₋₄alkyl, and 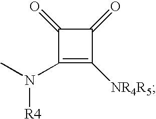
and
X is C═O;
or a pharmaceutically acceptable salt thereof.

8. A method according to claim 7 wherein the compound is selected from the group consisting of:
N-(2-Hydroxyl-3-aminosulfonyl-4-chlorophenyl)-N′-(2-bromophenyl) urea;
N-(2-Hydroxy-3-aminosulfonyl-4-chlorophenyl)-N′-(2,3-dichlorophenyl) urea;
N-[2-Hydroxy-3-(N″,N″-dimethyl)-aminosulfonyl4-chlorophenyl]-N′-(2,3-dichlorophenyl) urea;
N-(2-Hydroxy-3-N″,N″-dimethyl)-aminosulfonyl-4-chlorophenyl)-N′-(2-bromophenyl) urea;
N-(2-Hydroxy-3-N″-methylaminosulfonyl4-chlorophenyl)-N′-(2-bromophenyl) urea;
N-(2-Hydroxy-3-N″-methylaminosulfonyl4-chlorophenyl)-N′-(2,3-dichlorophenyl) urea;
N-[4-chloro-2-hydroxy-3-[N″-(2-methoxyethyl)aminosulfonyl]phenyl]-N′-(2,3-dichlorophenyl) urea;
1-(4-Chloro-2-hydroxy-3-methanesulfonyl-phenyl)-3-(2,3-dichloro-phenyl)-urea;
1-(2-Bromo-phenyl)-3-(4-cyano-2-hydroxy-3-methanesulfonyl-phenyl)-urea;
1-(2-Bromo-phenyl)-3-(4-cyano-2-hydroxy-3-propyl-phenyl)-urea;
1-(2-Bromo-phenyl)-3-[4-cyano-2-hydroxy-3-(1-methyl-butyl)-phenyl]-urea;
1-(2-Bromo-phenyl)-3-(4-cyano-2-hydroxy-3-isobutyl-phenyl)-urea;
1-(3-Bromo-4-cyano-2-hydroxy-phenyl)-3-(2-bromo-phenyl)-urea;
1-(4-Chloro-2-hydroxy-3-methanesulfinyl-phenyl)-3-(2,3-dichloro-phenyl)-urea;
{6-Chloro-3-[3-(2,3-dichloro-phenyl)-ureido]-2-hydroxy-phenyl }-methanesulfonamide;
3-[3-(2-Bromo-phenyl)-ureido]-6-chloro-2-hydroxy-benzamide;
6-Chloro-3-[3-(2,3-dichloro-phenyl)-ureido]-2-hydroxy-N-phenyl-benzamide;
1-[4-Chloro-2-hydroxy-3-(1-morpholin-4-yl-methanoyl)-phenyl]-3-(2,3-dichloro-phenyl)-urea;
6-Chloro-3-(3,4-dioxo-2-phenylamino-cyclobut-1-enylamino)-2-hydroxy-benzenesulfonamide;
3-(3,4-Dioxo-2-phenylamino-cyclobut-1-enylamino)-2-hydroxy-benzonitrile;
3-(3-Fluoro-2-hydroxy-phenylamino)-4-phenylamino-cyclobut-3-ene-1,2-dione;
4-(3,4-Dioxo-2-phenylamino-cyclobut-1-enylamino)-3-hydroxy-benzonitrile; and
3-(2-Hydroxy-4-nitro-phenylamino)-4-phenylamino-cyclobut-3-ene-1,2-dione.

9. A method of synthesizing a phenol-containing compound according to claim 3 comprising the steps of converting an aryl chloride according to formula (IV) 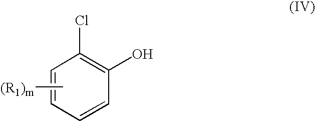
to a thiol according to formula (V): 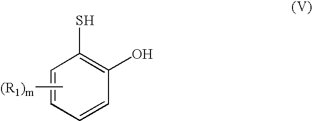
wherein R₁ is independently selected from the group consisting of hydrogen, halogen, nitro, cyano, S(O)ₜR₄, (CR₈R₈)_{q} S(O)ₜR₄, hydroxy substituted C₁₋₄ alkyl, heteroaryl, heteroaryl alkyl, heteroaryl C₂₋₁₀ alkenyl, C(O)NR₄R₅, C(O)OH, C(O)ORₐ, NR₄C(O)NR₄R₅, NR₄C(S)NR₄R₅, (CR₈R₈)_{q}C(O)NR₄R₅, (CR₈R₈)_{q} C(O)NR₄R₁₀, S(O)₃R₈, (CR₈R₈)_{q} C(O)R₁₁, (CR₈R₈)_{q} C(O)OR₁₁, (CR₈R₈)_{q} OC(O)R₁₁, (CR₈R₈)_{q}NR₄C(O)R₁₁, (CR₈R₈)_{q} C(NR₄)NR₄R₅, (CR₈R₈)_{q} NR₄C(NR₅)R₁₁, (CR₈R₈)_{q} NHS(O)₂R₁₃, (CR₈R₈)_{q} S(O)₂NR₄R₅, and 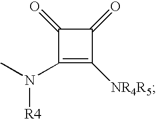
and
m is an integer from 1 to 4.

10. A method of synthesizing a phenol-containing compound according to claim 3 comprising the step of converting an aniline according to formula (VI) 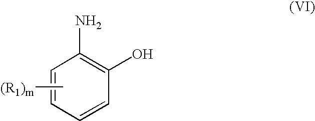
to a thiol according to formula (VII) 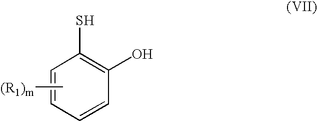
wherein R₁ is independently selected from the group consisting of hydrogen, halogen, nitro, cyano, S(O)ₜR₄, (CR₈R₈)_{q} S(O)ₜR₄, hydroxy substituted C₁₋₄ alkyl, heteroaryl, heteroaryl alkyl, heteroaryl C₂₋₁₀ alkenyl, C(O)NR₄R₅, C(O)OH, C(O)ORₐ, NR₄C(O)NR₄R₅, NR₄C(S)NR₄R₅, (CR₈R₈)_{q}C(O)NR₄R₅, (CR₈R₈)_{q} C(O)NR₄R₁₀, S(O)₃R₈, (CR₈R₈)_{q} C(O)R₁₁, (CR₈R₈)_{q} C(O)OR₁₁, (CR₈R₈)_{q} OC(O)R₁₁, (CR₈R₈)_{q}NR₄C(O)R₁₁, (CR₈R₈)_{q} C(NR₄)NR₄R₅, (CR₈R₈)_{q} NR₄C(NR₅)R₁₁, (CR₈R₈)_{q} NHS(O)₂R₁₃, (CR₈R₈)_{q} S(O)₂NR₄R₅, and 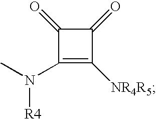
and
m is an integer from 1 to 4.
